(19) United States
(12) Patent Application Publication
(10) Pub. No.: US 2026/0201665 A1
(43) Pub. Date: Jul. 16, 2026

(54) METHODS FOR RECONSTRUCTING WATER-RETAINING AND WATER-BLOCKING LAYER BY GROUTING IN MINING AREA AND RELATED DEVICES

(71) Applicants: CHINA UNIVERSITY OF MINING AND TECHNOLOGY (BEIJING) INNER MONGOLIA RESEARCH INSTITUTE, Ordos (CN); CHINA UNIVERSITY OF MINING AND TECHNOLOGY, BEIJING, Beijing (CN)

(72) Inventors: Yifan ZENG, Beijing (CN); Aoshuang MEI, Beijing (CN); Qiang WU, Beijing (CN); Fulong ZHAI, Beijing (CN); Peng LI, Beijing (CN); Xinjiang WEI, Beijing (CN); Shi WANG, Beijing (CN); Xin WU, Beijing (CN); Yidi ZHANG, Beijing (CN)

(73) Assignees: CHINA UNIVERSITY OF MINING AND TECHNOLOGY (BEIJING) INNER MONGOLIA RESEARCH INSTITUTE, Ordos (CN); CHINA UNIVERSITY OF MINING AND TECHNOLOGY, BEIJING, Beijing (CN)

(21) Appl. No.: 19/378,292

(22) Filed: Nov. 3, 2025

(30) Foreign Application Priority Data
Jan. 13, 2025 (CN) 202510050728.3

(51) Int. Cl. E02D 3/12

(52) U.S. Cl. CPC E02D 3/12

(57) ABSTRACT

Provided is a method for reconstructing a water-retaining and water-blocking layer by grouting in a mining area, including: performing geophysical exploration within a space from an aquifer to a goaf in a coal mining remediation zone of a mining area to obtain a first apparent resistivity at each of positions within the space, and selecting positions within the space where the first apparent resistivity is less than or equal to a first preset resistivity to form a first dominant water-conducting channel zone; performing a water flow direction test in at least two monitoring wells within the coalmining remediation zone to obtain a first flow direction fitting function for each monitoring well, and selecting two first flow direction fitting functions to calculate a first flow direction intersection; and determining that the first dominant water-conducting channel zone is accurate and performing a grouting operation on the first dominant water-conducting channel zone.

### CROSS-REFERENCE TO RELATED APPLICATIONS

This application claims priority to the Chinese Patent Application No. 202510050728.3, filed on Jan. 13, 2025, the entire contents of which are incorporated herein by reference.

### TECHNICAL FIELD

The present disclosure relates to the field of grouting remediation technology, and in particular to a method for reconstructing a water-retaining and water-blocking layer by grouting in a mining area and a related device.

### BACKGROUND

Coal is an important natural resource. When the coal mining method involves longwall retreat comprehensive mechanized mining with full-seam mining technology and uses the complete caving method for roof management, if the thickness of the water-blocking soil layer beneath the bedrock and the loose layer is insufficient, the water-conducting fracture zone may directly connect to the weathered bedrock or even the loose layer in certain sections. This leads to a series of problems, including a gradual decline in the water level of the Quaternary superficial loose layer, the disappearance of surface runoff, and regional ecological degradation, with the mining progress.

To achieve water-retaining coal mining, it is necessary to reconstruct a water-blocking layer by grouting below the Quaternary loose layer. However, conventional remediation methods involve grouting the entire soil layer missing area and weak area. The large grouting volume and drilling workload, on the one hand, may easily impact groundwater quality and the environment. On the other hand, it consumes significant manpower and material resources, resulting in high costs. Therefore, there is an urgent need for a more efficient method for reconstructing a water-retaining and water-blocking layer by grouting in a mining area and a related device.

### SUMMARY

In view of the above, an objective of the present disclosure is to propose a method for reconstructing a water-retaining and water-blocking layer by grouting in a mining area and a related device, to solve the above technical problems.

One or more embodiments of the present disclosure provide a method for reconstructing a water-retaining and water-blocking layer by grouting in a mining area, comprising: performing geophysical exploration within a space from an aquifer to a goaf in a coal mining remediation zone of the mining area to obtain a first apparent resistivity at each of positions within the space, and selecting positions within the space where the first apparent resistivity is less than or equal to a first preset resistivity to form a first dominant water-conducting channel zone; performing a water flow direction test in at least two monitoring wells within the coal mining remediation zone to obtain a first flow direction fitting function for each monitoring well of the at least two monitoring wells, and selecting two first flow direction fitting functions to calculate a first flow direction intersection corresponding to two monitoring wells; and determining that the first dominant water-conducting channel zone is accurate when the first flow direction intersection is located within the first dominant water-conducting channel zone, and performing a grouting operation on the first dominant water-conducting channel zone.

One or more embodiments of the present disclosure provide a device for reconstructing a water-retaining and water-blocking layer by grouting in a mining area, comprising: a geophysical exploration module, configured to perform geophysical exploration from an aquifer to a goaf in a coal mining remediation zone of the mining area to obtain a first apparent resistivity at each of positions within a space, and to select positions within the space where the first apparent resistivity is less than or equal to a first preset resistivity to form a first dominant water-conducting channel zone; a well logging module, configured to perform a water flow direction test in at least two monitoring wells within the coal mining remediation zone to obtain a first flow direction fitting function for each monitoring well of the at least two monitoring wells, and to select two first flow direction fitting functions to calculate a first flow direction intersection corresponding to two monitoring wells; and a grouting module, configured to determine that the first dominant water-conducting channel zone is accurate when the first flow direction intersection is located within the first dominant water-conducting channel zone, and perform a grouting operation on the first dominant water-conducting channel zone.

One or more embodiments of the present disclosure provide an electronic device, comprising a memory, a processor, and a computer program stored in the memory and executable on the processor, wherein the processor, when executing the computer program, implements the above method for reconstructing the water-retaining and water-blocking layer by grouting in the mining area, the method including: performing geophysical exploration within a space from an aquifer to a goaf in a coal mining remediation zone of the mining area to obtain a first apparent resistivity at each of positions within the space, and selecting positions within the space where the first apparent resistivity is less than or equal to a first preset resistivity to form a first dominant water-conducting channel zone; performing a water flow direction test in at least two monitoring wells within the coal mining remediation zone to obtain a first flow direction fitting function for each monitoring well of the at least two monitoring wells, and selecting two first flow direction fitting functions to calculate a first flow direction intersection corresponding to two monitoring wells; and determining that the first dominant water-conducting channel zone is accurate when the first flow direction intersection is located within the first dominant water-conducting channel zone, and performing a grouting operation on the first dominant water-conducting channel zone.

### BRIEF DESCRIPTION OF THE DRAWINGS

To more clearly illustrate the technical solutions in the present disclosure or the related art, the following will briefly introduce the drawings required for describing the embodiments or the related art. Obviously, the drawings in the following description are merely embodiments of the present disclosure. For those of ordinary skill in the art, other drawings may be obtained based on these drawings without creative effort.

FIG. 1 is a schematic flowchart illustrating a process for reconstructing a water-retaining and water-blocking layer by grouting in a mining area according to some embodiments of the present disclosure.

FIG. 2 is a radar chart illustrating a water flow direction test according to some embodiments of the present disclosure.

FIG. 3 is a schematic flowchart illustrating a process for performing a grouting operation according to some embodiments of the present disclosure.

FIG. 4 is a schematic flowchart illustrating a process for performing a grouting operation according to other embodiments of the present disclosure.

FIG. 5 is a schematic diagram illustrating a process for determining a grouting stop time according to other embodiments of the present disclosure.

FIG. 6 is a schematic diagram illustrating a structure of a device for reconstructing a water-retaining and water-blocking layer by grouting in a mining area according to some embodiments of the present disclosure.

FIG. 7 is a schematic diagram illustrating a structure of an electronic device according to some embodiments of the present disclosure.

### DETAILED DESCRIPTION

To make the objectives, technical solutions, and advantages of the present disclosure clearer, the present disclosure is described in further detail below with reference to specific embodiments and the accompanying drawings.

It should be noted that, unless otherwise defined, technical or scientific terms used in the embodiments of the present disclosure should have the ordinary meanings as understood by those with general skill in the art. The terms “first”, “second”, and similar words used in the embodiments of the present disclosure do not denote any order, quantity, or importance, but are merely used to distinguish different components. Terms such as “include” or “comprise” mean that the elements or items preceding the word cover the elements or items listed after the word and their equivalents, without excluding other elements or items. Terms such as “connect” or “couple” are not limited to physical or mechanical connections, but may include electrical connections, whether direct or indirect. Terms such as “upper”, “lower”, “left”, and “right” are only used to indicate relative positional relationships. When the absolute position of the described object changes, the relative positional relationship may change accordingly.

Coal is an important natural resource. When the coal mining method involves longwall retreat comprehensive mechanized mining with full-seam mining technology and uses the complete caving method for roof management, if the thickness of the water-blocking soil layer beneath the bedrock and the loose layer is insufficient, the water-conducting fracture zone may directly connect to the weathered bedrock or even the loose layer in certain sections. This leads to a series of problems, including a gradual decline in the water level of the Quaternary superficial loose layer, the disappearance of surface runoff, and regional ecological degradation, with the mining progress. To achieve water-retaining coal mining, it is necessary to reconstruct a water-blocking layer by grouting below the Quaternary loose layer. However, conventional remediation methods involve grouting the entire soil layer missing area and weak area. The large grouting volume and drilling workload, on the one hand, may easily impact groundwater quality and the environment. On the other hand, it consumes significant manpower and material resources, resulting in high costs. Therefore, there is an urgent need for a more efficient method for reconstructing a water-retaining and water-blocking layer by grouting in a mining area.

Based on the above situation, a horizontal water-blocking layer can be constructed by grouting at the dominant water-conducting channels within the weathered bedrock stratum. This cuts off the recharge of Quaternary water to mine water inflow, thereby achieving water preservation for the Quaternary aquifer. The usual approach is to reconstruct the water-blocking layer by grouting the entire area. This manner undoubtedly increases costs. Furthermore, due to the influence of complex geological stresses, the difficulty of construction over the entire area also increases. A dominant water-conducting channel (also referred to as a dominant flow channel) refers to a path through which water flows easily.

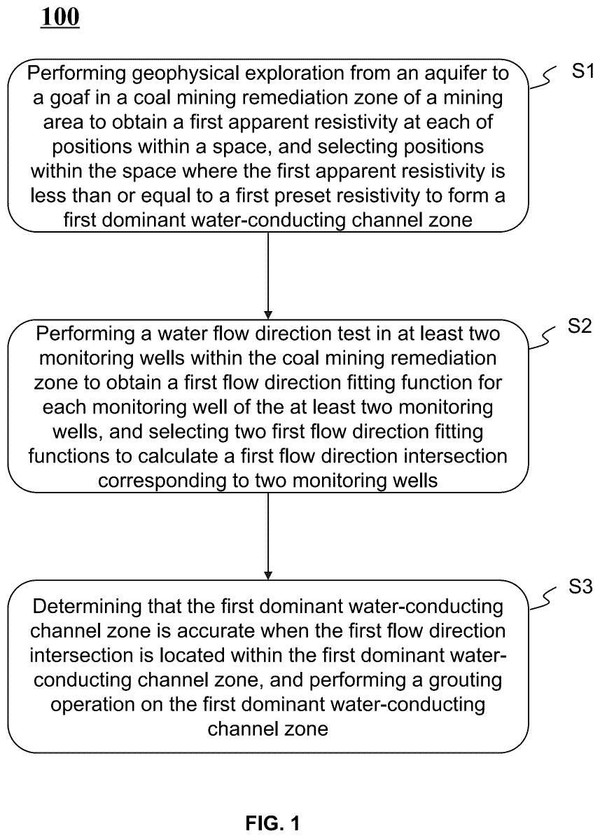

Therefore, how to accurately select the area where the dominant water-conducting channels are located for water-blocking layer reconstruction is a problem that urgently needs to be solved. Hereinafter, the technical solutions of the present disclosure are described in detail through specific embodiments and with reference to FIG. 1 to FIG. 7.

FIG. 1 is a schematic flowchart illustrating a process for reconstructing a water-retaining and water-blocking layer by grouting in a mining area according to some embodiments of the present disclosure.

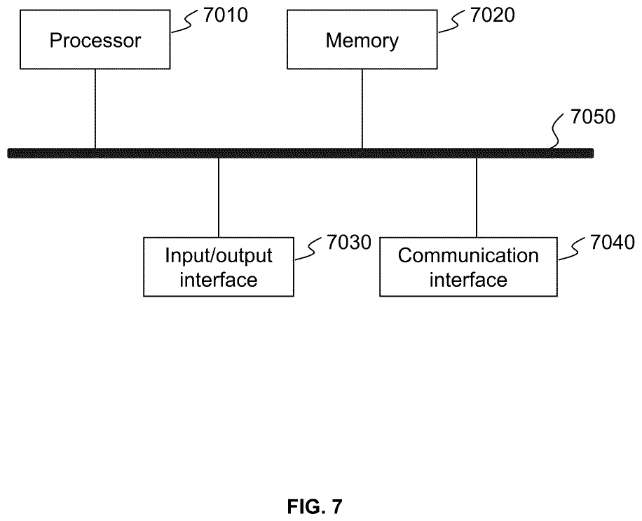

Some embodiments of the present disclosure provide a method for reconstructing a water-retaining and water-blocking layer by grouting in a mining area (also referred to as a method for water preservation by grouting in a coal mining remediation zone). The method is executed by a processor. For more information about the processor, refer to FIG. 7 and the related description. As shown in FIG. 1, the process 100 includes steps S1 to S3.

In S1, performing geophysical exploration within a space from an aquifer to a goaf in a coal mining remediation zone of the mining area to obtain a first apparent resistivity at each of positions within the space, and selecting positions within the space where the first apparent resistivity is less than or equal to a first preset resistivity to form a first dominant water-conducting channel zone.

The coal mining remediation zone refers to an area with missing or weak soil layers. The coal mining remediation zone may be obtained based on geological parameters before and after mining.

Geophysical exploration is abbreviated as GE. The geophysical exploration refers to an exploration manner that detects stratum lithology, geological structures, and other geological conditions by studying and observing changes in various geophysical fields. As an example, the geophysical exploration may employ the Time Domain Electromagnetic Method (TEM), etc.

The TEM is an effective way for detecting underground low-resistivity bodies (such as aquifers and water-rich zones), operating based on the principle of electromagnetic induction. The TEM operates by transmitting a horizontal transient electromagnetic field and observing its propagation and attenuation characteristics in the underground medium to infer the electrical characteristics of the underground medium, thereby analyzing the distribution and water abundance of groundwater.

The transient electromagnetic algorithm may accurately capture core information such as the distribution of underground aquifers related to water-filling sources, the relative differences in water abundance, and the spatial morphology of abnormal areas. This benefits from the significant electrical difference between the water-rich zones and the surrounding strata, i.e., the apparent resistivity of the water-rich zones is lower.

By measuring with the TEM, a first apparent resistivity of the underground medium may be obtained. The first apparent resistivity may be used to plot apparent resistivity profile maps or slice maps, intuitively revealing the distribution characteristics of the water-rich zones. A lower first apparent resistivity indicates better flow efficiency and less obstruction.

The underground medium refers to all substances within a certain range below the ground surface. For example, the underground medium may include solid media, liquid media, etc. Solid media may include rock or soil, etc. Liquid media may include groundwater, grout, etc.

The electrical characteristics of the underground medium refer to the physical properties exhibited by underground substances under the action of an electric field. For example, the electrical characteristics may include conductivity, dielectric properties, piezoelectric properties, etc.

In some embodiments, to achieve precise identification of the water-filling source in the coal mining remediation zone and rational planning of the grouting path, the method for reconstructing a water-retaining and water-blocking layer by grouting in a mining area further includes the following steps S101 to S102.

In S101, obtaining geological parameters of the coal mining remediation zone, and determining a water-filling source based on the geological parameters.

The geological parameters include stratum type, stratum thickness, stratum distribution state, geological structural features (such as faults, folds), buried depth of the groundwater level, aquifer permeability coefficient, hydraulic connection between aquifers, development height of the water-conducting fracture zone, etc.

The range of the stratum weak area and missing area may be judged based on the stratum thickness. The stratum thickness in the weak area is less than the conventional stable soil layer thickness, which can be screened by setting a threshold according to engineering needs. The stratum thickness in the missing area is 0. The range of the stratum weak area and missing area is the range of the coal mining remediation zone.

The water-filling source refers to the origin of mine water, mainly including atmospheric precipitation, ground surface water, groundwater, and old goaf water, etc. Based on parameters such as the aquifer permeability coefficient, the hydraulic connection between aquifers, and the development height of the water-conducting fracture zone, the origin of the water-filling source may be determined using conventional tracing algorithms, etc. Conventional tracing algorithms include hydrochemical fingerprint analysis, environmental isotope tracing, etc.

Taking a coal mine in the western region as an example, its coal-bearing strata belong to the Middle Jurassic Yan'an Formation. The coexistence characteristic of the coal seam and the aquifers is that the coal seam is below, and the aquifers are above. The aquifers within the mine field, from top to bottom, are a Quaternary phreatic aquifer, a Jurassic weathered bedrock aquifer, and a Jurassic bedrock aquifer. The water-blocking layer is mainly the Neogene soil water-blocking layer (with some missing areas). The currently mainly mined coal seam is the 3-1 coal seam of the Jurassic Yan'an Formation. The water-conducting fracture zone generated by the mining has developed into the weathered bedrock aquifer, resulting in the groundwater from the weathered bedrock of the coal seam roof and the Quaternary phreatic water becoming the main water-filling sources in the soil layer weak areas and missing areas.

In S102, performing geophysical exploration from a ground surface to the goaf in the coal mining remediation zone when the water-filling source is from a Quaternary aquifer.

The Quaternary aquifer refers to a groundwater layer contained in loose sediments formed during the Quaternary period.

When the water-filling source is from the Quaternary aquifer, it indicates a situation of water resource loss. In this case, the subsequent selection of the first dominant water-conducting channel zone and the grouting operation are necessary.

The apparent resistivity refers to a parameter reflecting the conductivity of the stratum. As an example, the apparent resistivity includes the first apparent resistivity and a second apparent resistivity.

The first apparent resistivity refers to a parameter reflecting the strength of stratum conductivity measured before completing the grouting operation.

For a description of the second apparent resistivity, please refer to the later sections and the related descriptions.

In some embodiments, a technical professional may determine the first apparent resistivity through geophysical exploration. For example, the first apparent resistivity is determined through a transient electromagnetic method or a high-density resistivity method.

A space from the aquifer to the goaf in the coal mining remediation zone may be represented asA=cf(X, Y, Z), where c denotes a selection coefficient. The selection coefficient refers to an adjustment coefficient preset based on geological conditions, engineering experience, or remediation objectives, f denotes a function describing spatial positions, which may be determined by fitting historical data, X, Y, and Z denote an abscissa, an ordinate, and an elevation, respectively, of corresponding positions within the space.

The first dominant water-conducting channel zone refers to an area in the stratum where water flow is prone to loss, as determined by geophysical exploration before the grouting operation.

In some embodiments, positions within the space where the first apparent resistivity is less than or equal to the first preset resistivity may be selected to form the first dominant water-conducting channel zone. The first preset resistivity is preset and obtained by a technical professional. For example, a range of the first preset resistivity is from 20 Ω·m to 40 Ω·m.

In some embodiments, for positions where the first apparent resistivity is less than or equal to the first preset resistivity, c is 1. For positions where the first apparent resistivity is greater than the first preset resistivity, c is 0. Then, a set A1 where c is 1 is the first dominant water-conducting channel zone. The set A1 represents positions where water flows more easily. These positions readily guide water from the aquifer to the goaf. Therefore, grouting and sealing operations need to be focused on this area for water retention.

In S2, performing a water flow direction test in at least two monitoring wells within the coal mining remediation zone to obtain a first flow direction fitting function for each monitoring well of the at least two monitoring wells, and select two first flow direction fitting functions to calculate a first flow direction intersection corresponding to two monitoring wells.

Although the test range of the geophysical exploration is relatively wide and the cost is low, the accuracy is relatively low. Based on this, the water flow direction test may be performed to further verify whether the aforementioned first dominant water-conducting channel zone is accurate. Technology for flow direction and velocity logging has high accuracy, but the cost is also relatively high, so it is unsuitable for high-density applications.

A monitoring well refers to a borehole used for monitoring subsurface physical and hydrological parameters. For example, the monitoring well includes an existing hydrological hole, a supplementary exploration hole, or a new borehole set up at a later stage, etc.

The water flow direction test refers to a manner for measuring a subsurface water flow direction and velocity through the monitoring well.

A flow direction and velocity logging device is used in combination with hydrological holes, supplementary exploration holes, etc., drilled in the early stage of construction in the construction area and its surroundings, which serve as monitoring wells, to perform the water flow direction test.

The first flow direction fitting function refers to a function obtained through the water flow direction test before the grouting operation, used to describe the subsurface water flow direction in a specific monitoring well.

The first flow direction intersection refers to a convergence point of subsurface water flow paths determined through the first flow direction fitting function.

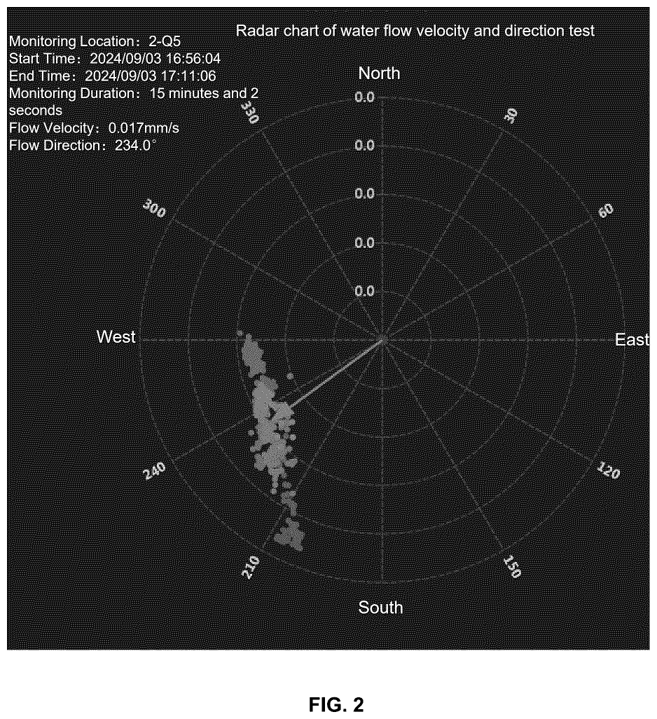

FIG. 2 is a radar chart illustrating the water flow direction test according to some embodiments of the present disclosure.

When performing the water flow direction test on the monitoring well, a measurement time corresponding to each point should be no less than 15 minutes. After the flow velocity and direction of the water flow stabilize, a radar chart for a certain stratum may be obtained, as shown in FIG. 2, for example. The first flow direction fitting function y=ax+m for the water flow direction in the monitoring well may be obtained through a conventional function fitting algorithm (e.g., a least squares method, Gaussian fitting), where x and y denote horizontal positions of a dominant water-conducting channel, a denotes a slope of the function fitted based on the radar chart, and m denotes an intercept of the function fitted based on the radar chart.

A flow direction is measured for another monitoring well, and a radar chart for the same stratum may be obtained. The first flow direction fitting function, y=bx+n, may be obtained by fitting a function using the same coordinate system. Similarly, b denotes a slope of the function fitted based on the radar chart, and n denotes an intercept of the function fitted based on the radar chart. Solving y=ax+m and y=bx+n yields (x1, y1). Then, considering the stratum where the test is conducted, i.e., a vertical position z1, a three-dimensional intersection (x1, y1, z1) may be obtained, which is the first flow direction intersection. This point is a convergence point of water flows from different positions, i.e., an actual dominant channel area in the aquifer.

In S3, determining that the first dominant water-conducting channel zone is accurate when the first flow direction intersection is located within the first dominant water-conducting channel zone, and performing a grouting operation on the first dominant water-conducting channel zone.

If (x1, y1, z1)∈A1, it may be proven that the first dominant water-conducting channel zone obtained by the geophysical exploration is accurate. Consequently, grouting remediation is performed only on the first dominant water-conducting channel zone to transform the weathered bedrock aquifer into a relatively water-blocking layer, thereby minimizing environmental impact and reducing economic costs to the greatest extent.

When the water flow direction test is performed on a plurality of monitoring wells, a plurality of first flow direction fitting functions may be obtained. Equation sets may be constructed pairwise to solve for corresponding first flow direction intersections. When all the first flow direction intersections are located within the first dominant water-conducting channel zone, the grouting operation is performed on the first dominant water-conducting channel zone. The accuracy is higher, but the cost also increases accordingly.

For the specific grouting operation, a detailed reconstruction plan needs to be formulated based on a scope of the water-blocking layer reconstruction and geological conditions. This includes selection of grouting materials, setting of grouting pressure, construction details of grouting holes, etc., which are not elaborated here.

The first dominant water-conducting channel zone may be preliminarily obtained through the geophysical exploration. Then, the first flow direction intersection may be obtained using the flow direction test to verify the first dominant water-conducting channel zone. When the first flow direction intersection is located within the first dominant water-conducting channel zone, it indicates that the first dominant water-conducting channel zone is relatively accurate. Performing the grouting operation only on the first dominant water-conducting channel zone may achieve the water retention effect in the coal mining remediation zone. This avoids blind construction in traditional manners, reduces unnecessary drilling and construction, lowers overall remediation costs, and, to some extent, reduces environmental impact.

The method for reconstructing a water-retaining and water-blocking layer by grouting in a mining area is simple and convenient. It can quickly and accurately achieve water retention by grouting, with low cost, effectively reducing the loss of shallow surface water resources. The combined application of the geophysical exploration technology and well logging technology enables rapid and accurate identification of water-rich zones and water abundance conditions in the remediation zone, avoiding blind construction in traditional manners. It reduces unnecessary drilling and construction, lowers overall remediation costs, and, to some extent, reduces environmental impact. A real-time monitoring system provides feedback, enabling dynamic adjustment of remediation measures to ensure long-term maintenance of the remediation effect.

In some embodiments, to achieve dynamic delineation of the first dominant water-conducting channel zone and ensure the effectiveness of the grouting operation, the method for reconstructing a water-retaining and water-blocking layer by grouting in a mining area further includes the following step S4:

In S4, increasing the first preset resistivity to reform the first dominant water-conducting channel zone when the first flow direction intersection is located outside the first dominant water-conducting channel zone, and subsequently using the water flow direction test to verify again whether the first dominant water-conducting channel zone is accurate.

A magnitude of the increase in the first preset resistivity may be set by a technical professional based on experience.

In some embodiments, when the first flow direction intersection is located outside the first dominant water-conducting channel zone, the geophysical exploration may also be reperformed again from the aquifer to the goaf in the coal mining remediation zone to obtain the first apparent resistivity at various positions within the space.

If (x1, y1, z1)∉A1, it indicates that the first dominant water-conducting channel zone obtained by the geophysical exploration is inaccurate. This inaccuracy may be caused by a low setting of the first preset resistivity or inaccuracy of the geophysical exploration. It is necessary to increase the first preset resistivity to reobtain the first dominant water-conducting channel zone, or reperform the geophysical exploration to obtain a new first apparent resistivity, and then reobtain the first dominant water-conducting channel zone. Subsequently, the same flow direction measurement manner is used again to verify whether the new first dominant water-conducting channel zone is accurate.

When the first flow direction intersection is located outside the first dominant water-conducting channel zone, the first dominant water-conducting channel zone is reformed by increasing the first preset resistivity, and the same water flow direction test manner is used for re-verification. This ensures that the finally determined first dominant water-conducting channel zone highly matches the actual hydrogeological conditions, effectively avoiding ineffective construction caused by misjudgment of the area, thereby improving grouting efficiency and success rate.

In some embodiments, to achieve quantitative evaluation of the grouting effect and intelligent determination of construction nodes, ensuring the economy of the water-blocking layer reconstruction, the method for reconstructing a water-retaining and water-blocking layer by grouting in a mining area further includes the following steps S501-S502:

In S501, after performing the grouting operation, performing geophysical exploration within the space again from the aquifer to the goaf in the coal mining remediation zone to obtain the second apparent resistivity at each of the positions within the space; selecting positions within the space where the second apparent resistivity is less than or equal to the first preset resistivity to form a second dominant water-conducting channel zone.

The second apparent resistivity refers to a parameter reflecting the strength of stratum conductivity measured after completing the grouting operation. An acquisition manner for the second apparent resistivity is similar to the manner for acquiring the first apparent resistivity described above.

The second dominant water-conducting channel zone refers to an area in the stratum where there is still a risk of water leakage, as measured again by the geophysical exploration after the grouting operation.

After the grouting operation, to inspect the grouting effect, the geophysical exploration may be performed again to obtain the second dominant water-conducting channel zone A2 according to the same first preset resistivity. The manner is the same as the manner for obtaining the first dominant water-conducting channel zone described above.

In S502, stopping grouting when the second dominant water-conducting channel zone is located within the first dominant water-conducting channel zone.

It is determined whether the second dominant water-conducting channel zone is located within the first dominant water-conducting channel zone by determining whether coordinates of each position in the second dominant water-conducting channel zone fall within a coordinate range of the first dominant water-conducting channel zone.

When the second dominant water-conducting channel zone is located within the first dominant water-conducting channel zone, it indicates that the original first dominant water-conducting channel zone has become smaller, demonstrating that the sealing effect of the grouting is effective, and grouting may be stopped. For more manners regarding stopping grouting, refer to the following section and the related descriptions.

By comparing the positional relationship between the second dominant water-conducting channel zone after grouting and the first dominant water-conducting channel zone before grouting, precise determination of the grouting effect is achieved. Grouting is stopped when the second zone is completely located within the first zone, ensuring that the water-blocking layer reconstruction meets standards and avoiding ineffective construction.

In some embodiments, to achieve preliminary quantification and definition of potential dominant water-conducting areas in the aquifer, providing baseline data for subsequent grouting effect evaluation, after obtaining the first apparent resistivity at various positions within the space, the following step S111 is included:

In S111, selecting positions within the aquifer where the first apparent resistivity is less than or equal to the first preset resistivity to form a first zone, an area of the first zone being a first area.

Positions in the aquifer where the first apparent resistivity is less than or equal to the first preset resistivity are selected to form the first zone. The area of the first zone is referred to as the first area, denoted as s1, which facilitates subsequent inspection of the grouting effect.

In some embodiments, to achieve quantitative evaluation of the grouting effect and precise control of construction nodes, the method for reconstructing a water-retaining and water-blocking layer by grouting in a mining area further includes the following steps S511 and S512.

In S511, after performing the grouting operation, performing the geophysical exploration again from the aquifer to the goaf in the coal mining remediation zone to obtain a second apparent resistivity at each of the positions within the space; selecting positions within the aquifer where the second apparent resistivity is less than or equal to the first preset resistivity to form a second zone, an area of the second zone being a second area.

After the grouting operation, to inspect the grouting effect, the geophysical exploration may be performed again. The second zone in the aquifer may be obtained according to the same first preset resistivity, using a manner identical to the aforementioned manner for obtaining the first zone. The area of the second zone is referred to as the second area, denoted as s2.

In S512, stopping grouting when the second area is greater than the first area.

Similarly, when s2>s1, it indicates that the effective area of the aquifer has increased, demonstrating that the sealing effect of the grouting is effective, and grouting may be stopped.

By comparing the sizes of the first area before grouting and the second area after grouting, quantitative evaluation of the grouting effect is achieved. When the second area is greater than the first area, grouting is stopped promptly, effectively avoiding over-construction and improving project economy and safety.

In some embodiments, to monitor and determine changes in the groundwater flow field after grouting, the method for reconstructing a water-retaining and water-blocking layer by grouting in a mining area further includes the following steps S521 and S522.

In S521, after performing the grouting operation, performing the water flow direction test again in the at least two monitoring wells within the coal mining remediation zone to obtain a second flow direction fitting function for the each monitoring well, and selecting two second flow direction fitting functions to calculate a second flow direction intersection corresponding to two monitoring wells.

The second flow direction fitting function refers to a function obtained through the water flow direction test after the grouting operation, used to describe the subsurface water flow direction in a specific monitoring well.

The second flow direction intersection refers to a convergence point of subsurface water flow paths determined through the second flow direction fitting function.

After the grouting operation, to inspect the grouting effect, the water flow direction test may be performed again to obtain the second flow direction intersection (x2, y2, z2), using a manner identical to the aforementioned manner for obtaining the first flow direction intersection (x1, y1, z1).

In S522, stopping grouting when the second flow direction intersection is located outside the first dominant water-conducting channel zone.

If (x2, y2, z2)∉A1, or if the second flow direction intersection has no solution, it indicates that the original intersection point no longer exists, demonstrating that the sealing effect of the grouting is effective, and grouting may be stopped.

By performing the water flow direction test again after grouting and obtaining the second flow direction intersection, dynamic monitoring and determination of the grouting effect may be achieved. When the second flow direction intersection is located outside the first dominant water-conducting channel zone, grouting is stopped, ensuring effective reconstruction of the water-blocking layer and avoiding over-construction.

In some embodiments, to achieve multi-dimensional and comprehensive precise determination of the grouting effect, the method for reconstructing a water-retaining and water-blocking layer by grouting in a mining area further includes the following step S531.

In S531, when the second area is greater than the first area, a volume of the second dominant water-conducting channel zone is less than or equal to a volume of the first dominant water-conducting channel zone multiplied by a first preset ratio, and a second flow direction intersection is located outside the first dominant water-conducting channel zone, stopping grouting.

dominant water-conducting channel zonedominant water-conducting channel zonedominant water-conducting channel zonedominant water-conducting channel zone

To ensure the effectiveness of the grouting effect, the aforementioned three manners may be combined for joint judgment: the second area is greater than the first area, the second dominant water-conducting channel zone is located within the first dominant water-conducting channel zone, and the second flow direction intersection is located outside the first dominant water-conducting channel zone. This leads to the grouting stop manner corresponding to this embodiment. That is, when the second area is greater than the first area, the volume of the second dominant water-conducting channel zone is less than or equal to the volume of the first dominant water-conducting channel zone multiplied by the first preset ratio, and the second flow direction intersection is located outside the first dominant water-conducting channel zone. The first preset ratio may be preset. For example, when the first preset ratio is 5%, and s2>s1, A2≤5%×A1, and (x2, y2, z2)∉A1, it indicates an excellent water retention effect, and no further grouting is needed.

By comprehensively judging that the second area is greater than the first area, the volume of the second dominant water-conducting channel zone is less than or equal to the volume of the first dominant water-conducting channel zone multiplied by the first preset ratio, and the second flow direction intersection is located outside the first dominant water-conducting channel zone, the precise evaluation of the grouting effect and scientific stopping of grouting are further improved, effectively avoiding over-grouting and ensuring the reliability of the water-blocking layer reconstruction.

In some embodiments, to evaluate the water retention effect of the water-blocking layer, the method for reconstructing a water-retaining and water-blocking layer by grouting in a mining area further includes the following steps S601 and S602.

In S601, after a preset time period following completion of grouting, performing a water level test in a monitoring well within the coal mining remediation zone to obtain a first water level drop, and performing the water level test in a monitoring well outside the coal mining remediation zone to obtain a second water level drop.

The water level test refers to a test that measures changes in the groundwater level of the aquifer using a water level sensor in the monitoring well.

The first water level drop refers to a water level drop value measured in the monitoring well within the coal mining remediation zone. The second water level drop refers to a water level drop value measured in the monitoring well outside the coal mining remediation zone. In some embodiments, a professional technician evaluates the effect of the grouting operation by comparing the first water level drop and the second water level drop.

After grouting is completed, long-term observation holes or supplementary exploration holes may also be used as the monitoring wells to long-term inspect the water retention effect of the grouting. The preset time period may be determined by professional technicians according to requirements. For example, when the preset time period is 365 days, performing the water level test in the monitoring well within the coal mining remediation zone yields the first water level drop. The first water level drop may be obtained by determining the difference between the water level at the time of grouting completion and the water level one year later. Similarly, performing the water level test in the monitoring well outside the coal mining remediation zone yields the second water level drop, which may also be obtained by determining the difference between the water level at the time of grouting completion and the water level one year later.

In S602, completing water retention when the first water level drop is less than or equal to the second water level drop multiplied by a second preset ratio.

The second preset ratio may be preset. For example, when the second preset ratio is 30%, and the first water level drop is less than or equal to 30% of the second water level drop, it indicates that the water level drop in the coal mining remediation zone is very small, demonstrating that the water retention effect remains effective.

By comparing the water level drops inside and outside the coal mining remediation zone after grouting, and determining that water retention is completed when the first water level drop is less than or equal to the second water level drop multiplied by the second preset ratio, quantitative evaluation and scientific determination of the reconstruction effect of the water-blocking layer are achieved.

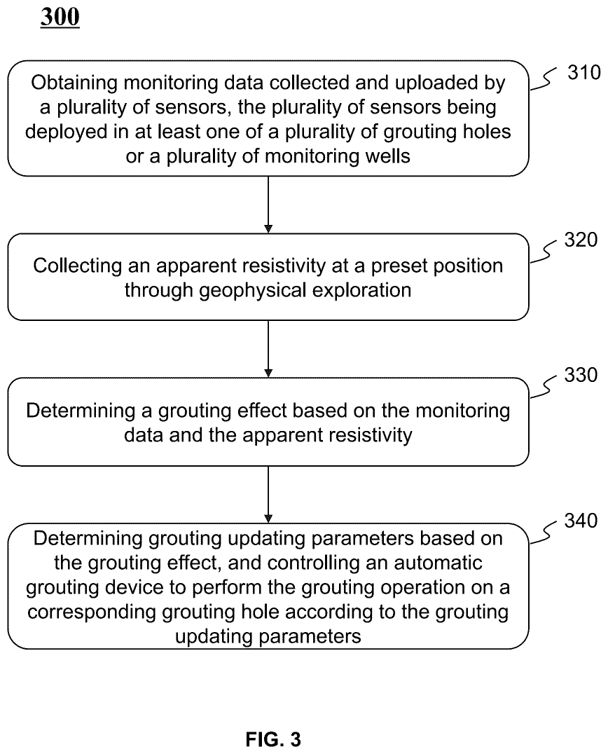

FIG. 3 is a schematic flowchart illustrating a process for performing a grouting operation according to some embodiments of the present disclosure.

As shown in FIG. 3, a process 300 includes the following steps, executed by a processor.

In step 310, obtaining monitoring data collected and uploaded by a plurality of sensors, the plurality of sensors being deployed in a plurality of grouting holes and/or the plurality of monitoring wells

For more descriptions regarding the monitoring wells, refer to FIG. 1 and the related description.

A sensor refers to a device used to collect physical parameters before and during grouting. For example, sensors include a flow velocity and direction sensor, a pore water pressure sensor, a grout rheology sensor, a resistivity sensor. The flow velocity and direction sensor is used to measure a flow velocity and a flow direction of grout or groundwater. The pore water pressure sensor is used to measure a pressure of grout or groundwater. The grout rheology sensor is used to measure flow characteristics of injected grout. The resistivity sensor is used to measure a resistivity of a stratum medium.

A grouting hole refers to a hole drilled for injecting grout into a stratum.

The monitoring data refers to data collected in real time by the aforementioned sensors, which may be divided into first monitoring data collected before grouting as a baseline and second monitoring data used for real-time monitoring during the grouting process. The first monitoring data includes a pore water pressure, a grout flow velocity, etc., collected before grouting. The second monitoring data includes the pore water pressure, the grout flow velocity, etc., collected in real time during the grouting process.

Before grouting, a sensor is installed in at least one of the grouting hole or the monitoring well to obtain first monitoring data in real time and upload the first monitoring data to a processor. During grouting, the sensor continuously collects the second monitoring data and uploads the second monitoring data to the processor in real time via wireless or wired communication. A data collection frequency may be set according to monitoring requirements.

In step 320, collecting an apparent resistivity at a preset position through the geophysical exploration

For more details about the geophysical exploration and the apparent resistivity, refer to FIG. 1 and the related description.

The preset position refers to a representative position where real-time geophysical exploration is performed during the grouting process. Collecting the apparent resistivity at the preset position helps ensure effectiveness while avoiding the time and effort required for large-scale geophysical exploration. For example, a position may be set every first preset distance along a flow direction based on an underground water flow direction monitored by the monitoring well. Alternatively, a plurality of positions may be set radially in a plurality of directions with a first flow direction intersection as a center. The first preset distance is set by a professional technician.

A geophysical exploration device (e.g., a transient electromagnetic instrument, a high-density resistivity instrument) is deployed at the preset position. The device transmits a transient electromagnetic field underground through an electromagnetic induction principle and receives an attenuation response signal from an underground medium. After data processing, apparent resistivity data is generated. The data collection frequency may be set according to a grouting progress, for example, once every 30 minutes.

In step 330, determining a grouting effect based on the monitoring data and the apparent resistivity

The grouting effect refers to a quantitative indicator used to evaluate an effectiveness of the grouting operation, reflecting a degree to which a grout seals a dominant water-conducting channel. The grouting effect may be generated by comprehensively analyzing the monitoring data and the apparent resistivity. For example, an effect value between 0 and 1 is generated, where a larger value indicates a better sealing effect.

In some embodiments, when a pore water pressure is higher than a water pressure threshold, a grout flow velocity is lower than a flow velocity threshold, and the apparent resistivity at the preset position is greater than a resistivity threshold are simultaneously satisfied, and it may be determined that the grouting has effectively sealed the channel.

The water pressure threshold, the flow velocity threshold, and the resistivity threshold may be determined based on historical data. For example, the historical data with working condition parameters (e.g., a monitoring well flow rate, a water level, and the first area) similar to current working conditions (where differences satisfy a difference condition), which have proven effective in sealing water-conducting channels, is screened out. A mean value of the pore water pressure, a mean value of the grout flow velocity, and a mean value of the apparent resistivity in the historical data are determined. The mean values are set as the corresponding water pressure threshold, the grout flow velocity threshold, and the resistivity threshold, respectively.

The difference condition may be preset.

In some embodiments, the processor generates the grouting effect based on the monitoring data and the apparent resistivity in various manners. For example, the processor may determine a ratio of the pore water pressure to the water pressure threshold, a ratio of the grout flow velocity threshold to the grout flow velocity, and a ratio of a real-time apparent resistivity to the resistivity threshold. An average value of the three ratios is then used as the grouting effect. Since the grouting effect is a value between 0 and 1, when the average value is greater than 1, the grouting effect is taken as 1.

In step 340, determining grouting updating parameters based on the grouting effect, and controlling an automatic grouting device to perform the grouting operation on a corresponding grouting hole according to the grouting updating parameters.

The grouting updating parameters refer to grouting parameters dynamically adjusted based on the grouting effect.

The grouting parameters refer to working parameters involved when the automatic grouting device performs the grouting operation. For example, the grouting parameters include a grouting pressure and a grouting speed.

The automatic grouting device refers to a device that performs the grouting operation, including a grouting pump, a control system, and a pipeline system. The grouting pump is configured to provide the grouting pressure and a grouting flow rate. The control system is configured to receive instructions from the processor and adjust the grouting parameters. The pipeline system is configured to deliver the grout to the grouting hole.

The grouting pressure refers to a pressure applied when injecting the grout into a stratum during the grouting operation.

The grouting speed refers to a speed at which the grout is injected into the stratum during the grouting operation.

In some embodiments, the processor may determine the grouting updating parameters in various ways. For example, the processor may determine the grouting updating parameters by querying a preset relationship table based on a current grouting effect.

In some embodiments, the preset relationship table may include a correspondence between the grouting effect and the grouting updating parameters. The preset relationship table may be determined based on the grouting effect and actual grouting updating parameters in the historical data.

In some embodiments, the processor may also determine the grouting updating parameters based on the grouting parameters and a comprehensive change rate using the following formula (1), exemplarily:

$${P}_{n e w} = P \times ( 1 - j ) , \qquad ( 1 ) ,$$

where in formula (1), P_{new} denotes the grouting updating parameter, P denotes the grouting parameter, and j denotes the comprehensive change rate.

The comprehensive change rate is used to reflect a change trend of a stratum resistance and may be characterized by a numerical value.

In some embodiments, the processor may determine the comprehensive change rate based on a positive correlation between the comprehensive change rate and a water pressure change rate and a flow velocity change rate, exemplarily:

$$j = {w}_{1} \times r + {w}_{2} \times t , \qquad ( 2 ) ,$$

where in formula (2), j denotes the comprehensive change rate, r denotes the water pressure change rate, t denotes the flow velocity change rate, and w₁ and w₂ denote weight coefficients that may be preset.

The water pressure change rate refers to a change amount of the pore water pressure in the stratum within a preset unit time period.

The flow velocity change rate refers to a change amount of the flow velocity of the grout within the preset unit time period.

The preset unit time period is preset by a professional technician.

In some embodiments, the processor sends the grouting updating parameters to the control system of the automatic grouting device. The control system adjusts a pressure and a flow rate of the grouting pump and injects the grout into a designated grouting hole according to the grouting updating parameters. During grouting, devices such as the sensor continuously collect data, forming a closed-loop control.

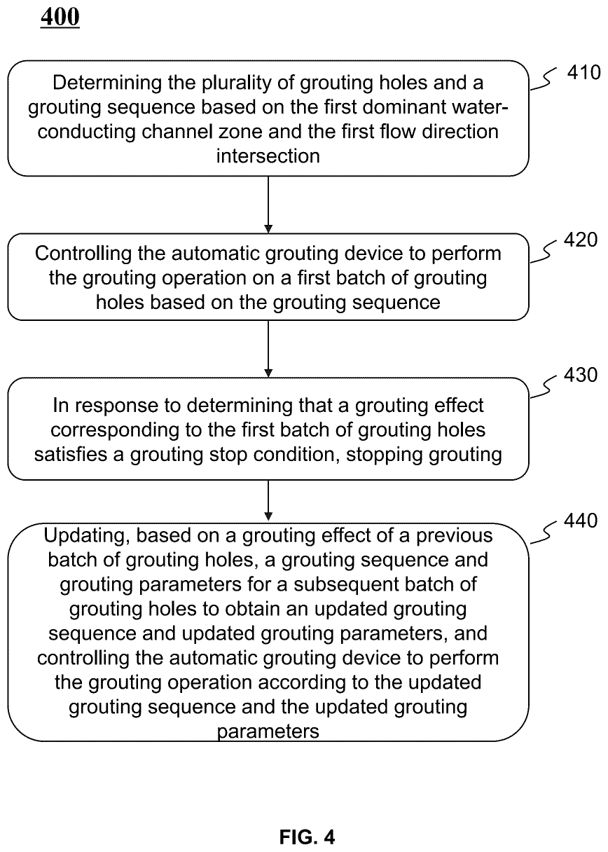

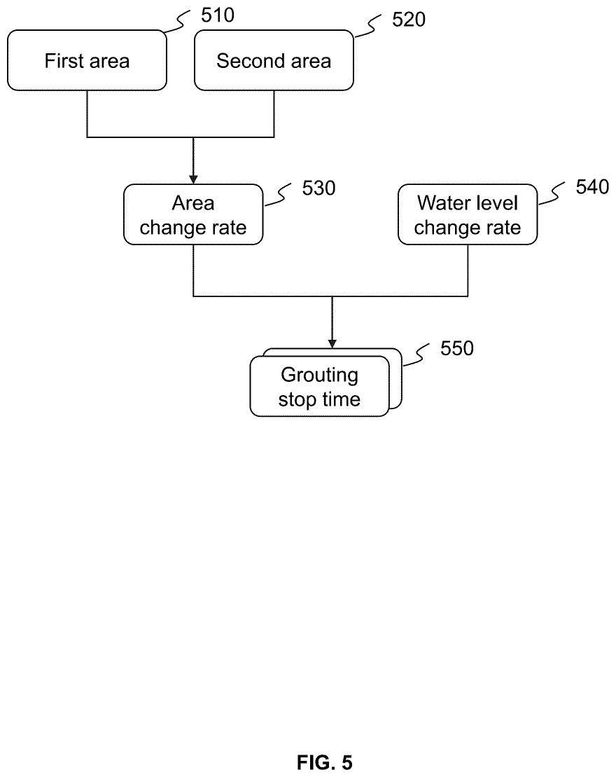

For more details about the grouting operation, refer to FIG. 1, FIG. 4, FIG. 5, and the related descriptions.

In some embodiments, the processor adjusts borehole positions of a subsequent batch of the grouting holes based on a difference between a characteristic of the second dominant water-conducting channel zone after performing the grouting operation using a previous batch of the grouting holes and a characteristic of the first dominant water-conducting channel zone.

The characteristic of the first dominant water-conducting channel zone (hereinafter referred to as a first characteristic) refers to a quantitative attribute of the dominant water-conducting channel zone before a first grouting operation, including area and spatial coordinates of the first dominant water-conducting channel zone. The characteristic of the second dominant water-conducting channel zone (hereinafter referred to as a second characteristic) refers to a corresponding quantitative attribute of the dominant water-conducting channel zone after the first grouting operation, and its parameter type is similar to that of the first characteristic.

The first characteristic and the second characteristic may be determined through experiments.

A difference refers to a measurable change in amount between the first characteristic and the second characteristic on the same quantitative parameter before and after the grouting operation. For example, the difference includes a boundary reduction value in a certain direction between the second dominant water-conducting channel zone and the first dominant water-conducting channel zone.

A boundary reduction value refers to a difference between a distance from a boundary of the first dominant water-conducting channel zone to its center point and a distance from a boundary of the second dominant water-conducting channel zone to its center point in a certain direction. The boundary reduction value may be used to determine a difference in the grouting effect in a certain direction.

The directions may include cardinal directions (e.g., east, west, south, north), a water flow direction monitored by the monitoring well before the current batch of grouting, etc. A certain direction is set by a professional technician.

In some embodiments, the processor may determine a center point of the first dominant water-conducting channel zone and a center point of the second dominant water-conducting channel zone, respectively. The center point may be determined by locating a spatial center of gravity or the like. It should be noted that the center point of the first dominant water-conducting channel zone and the center point of the second dominant water-conducting channel zone may not coincide due to the influence of grouting. Next, using the respective center points as origins, distances from boundaries of the two zones to their respective center points are measured in the same direction, designated as boundary distances. Finally, in the same direction, a difference between the boundary distance of the first dominant water-conducting channel zone and the boundary distance of the second dominant water-conducting channel zone is taken as the boundary reduction value corresponding to that direction.

The borehole position refers to a borehole point with specific spatial coordinates used for performing the grouting operation.

In some embodiments, the processor may adjust the borehole positions of the grouting holes in a subsequent batch based on the difference between the first characteristic and the second characteristic in various ways. For example, after completing the grouting operation of the previous batch, the second characteristic is obtained through the geophysical exploration and compared with the first characteristic before grouting to determine a boundary reduction value in at least one direction. If the boundary reduction value in a certain direction is less than or equal to a reduction threshold, it indicates that the grouting effect in that direction is poor. Therefore, the borehole positions for the subsequent batch will be preferentially adjusted towards that direction. For example, by reducing a clustering radius in cluster analysis to increase the density of subsequent grouting holes, targeted reinforcement grouting may be performed on weak sealing areas. The reduction threshold may be obtained by presetting.

By comparing the difference between the first characteristic and the second characteristic before and after grouting, a weak direction of the grouting operation is identified, thereby enabling real-time adjustment of the borehole positions for the grouting holes in the subsequent batches. This not only avoids blind construction and resource waste in traditional non-differentiated grouting operations but also effectively copes with complex geological conditions, improving the grouting success rate and reducing costs and construction cycles through real-time adjustments.

FIG. 4 is a schematic flowchart illustrating a process for performing the grouting operation according to other embodiments of the present disclosure.

As shown in FIG. 4, a process 400 includes the following steps, which are executed by the processor.

In step 410, determining the plurality of grouting holes and a grouting sequence based on the first dominant water-conducting channel zone and the first flow direction intersection.

For more descriptions regarding the first dominant water-conducting channel zone and the first flow direction intersection, please refer to FIG. 1 and the related descriptions.

The grouting sequence refers to an order in which the plurality of grouting holes undergo the grouting operation. For example, a first batch includes a grouting hole a1 and a grouting hole a3, and a second batch includes a grouting hole a2 and a grouting hole a4.

In some embodiments, the processor may arrange the grouting holes at intervals of a second preset distance annularly or radially around the first flow direction intersection as a center. Based on a distance between each grouting hole and the first flow direction intersection and a shortest distance between the each grouting hole and a boundary of the first dominant water-conducting channel zone, grouting holes satisfying conditions (e.g., the distance to the first flow direction intersection is less than a first threshold, and the shortest distance to the boundary of the first dominant water-conducting channel zone is greater than a second threshold) are selected as the grouting holes for a subsequent grouting operation. The second preset distance, the first threshold, and the second threshold may be set by a professional technician.

The distance between the each grouting hole and the first flow direction intersection refers to a Euclidean distance between three-dimensional coordinates of the grouting hole and three-dimensional coordinates of the first flow direction intersection.

The shortest distance refers to a minimum spatial distance from the grouting hole to the boundary of the first dominant water-conducting channel zone, obtained by determining distances from the grouting hole to various points on the zone boundary and taking the minimum value.

In some embodiments, the processor establishes a planar coordinate system with the first flow direction intersection as an origin and determines coordinates of the plurality of grouting holes. Then, based on a clustering radius, the plurality of grouting holes whose coordinate distance between any two is less than the clustering radius are grouped into a same cluster, thereby determining a plurality of clusters. The plurality of grouting holes corresponding to a same cluster center are taken as the grouting holes of a same batch. The grouting sequence for the grouting holes of different batches is determined according to distances from each cluster center to the first flow direction intersection from near to far. Manners for cluster analysis include K-means clustering, hierarchical clustering, etc.

Clustering features include a distance and an azimuth of each grouting hole relative to the first flow direction intersection.

The clustering radius is negatively correlated with an average value of a flow velocity of groundwater monitored in the monitoring well.

In some embodiments, the processor determines the first batch of grouting holes based on a distance between each grouting hole of the plurality of grouting holes and the first flow direction intersection and a shortest distance between the each grouting hole and a boundary of the first dominant water-conducting channel zone.

In some embodiments, a professional technician may also determine the first batch of grouting holes based on the first dominant water-conducting channel zone and the first flow direction intersection according to construction experience. For example, all the grouting holes that are within the first dominant water-conducting channel zone and whose distance to the first flow direction intersection is less than a preset distance threshold are determined as the first batch of grouting holes. The preset distance threshold is set by a technical professional.

By determining the first batch of grouting holes in advance, it is ensured that grout can quickly seal the main water-conducting channels, avoiding waste due to grout diffusion towards boundaries, and improving grouting efficiency and precision.

In step 420, controlling the automatic grouting device to perform the grouting operation on a first batch of grouting holes based on the grouting sequence.

In some embodiments, the processor sends coordinates and the grouting parameters corresponding to the first batch of grouting holes to the automatic grouting device based on the grouting sequence to perform the grouting operation on each grouting hole.

In some embodiments, it is known that there are two situations, i.e., that the grouting effect corresponding to the first batch of grouting holes satisfies a grouting stop condition and the grouting effect corresponding to the first batch of grouting holes does not satisfy the grouting stop condition. For a related description of satisfying the grouting stop condition, please refer to step 430. For a related description of not satisfying the grouting stop condition, please refer to step 440.

In step 430, in response to determining that a grouting effect corresponding to the first batch of grouting holes satisfies a grouting stop condition, stopping grouting.

As described in FIG. 1, the grouting stop condition include: (1) a second area is greater than a first area, (2) the second dominant water-conducting channel zone is located within the first dominant water-conducting channel zone, (3) the second flow direction intersection is located outside the first dominant water-conducting channel zone, (4) when the second area is greater than the first area, a volume of the second dominant water-conducting channel zone is less than or equal to a volume of the first dominant water-conducting channel zone multiplied by a first preset ratio, and the second flow direction intersection is located outside the first dominant water-conducting channel zone.

In some embodiments, when any grouting stop condition is satisfied, the processor sends a stop instruction to the automatic grouting device to terminate all the grouting operations, thereby saving significant time and cost.

In step 440, or, in response to determining that the grouting effect corresponding to the first batch of grouting holes does not satisfy the grouting stop condition, updating, based on a grouting effect of a previous batch of the grouting holes, the grouting sequence and the grouting parameters for the subsequent batch of the grouting holes to obtain an updated grouting sequence and the updated grouting parameters, and controlling the automatic grouting device to perform the grouting operation according to the updated grouting sequence and the updated grouting parameters.

For more descriptions regarding the grouting parameters and automatic grouting devices, refer to FIG. 3 and the related descriptions.

In some embodiments, the grouting sequence of the subsequent batch of the grouting holes is determined in a manner similar to the manner of determining the grouting sequence described in step 410. For example, the processor establishes a plane coordinate system based on the second flow direction intersection and re-determines coordinates of the plurality of grouting holes on which the grouting operation has not been performed. Then, based on a clustering radius, the processor classifies a plurality of grouting holes, among the grouting holes on which the grouting operation has not been performed, in which a coordinate distance between any two grouting holes is less than the clustering radius, into a same cluster, thereby determining a plurality of clusters. The plurality of grouting holes corresponding to a same cluster center serve as the grouting holes of a same batch. The grouting sequence corresponding to the subsequent batch of grouting holes is determined according to a distance from each cluster center to the first flow direction intersection from near to far.

For more descriptions regarding the clustering radius, please refer to step 410.

In some embodiments, the processor sends the updated grouting sequence and the updated grouting parameters to the automatic grouting device and performs the grouting operation until the grouting stop condition is satisfied.

Through batch-by-batch grouting and dynamic adjustment, the grouting operation is preferentially performed on the first batch of grouting holes to avoid ineffective construction. Meanwhile, a subsequent plan is optimized based on a real-time grouting effect, thereby improving grouting efficiency, reducing cost and construction cycle, and ensuring grouting accuracy under complex geological conditions.

FIG. 5 is a schematic diagram illustrating a process for determining the grouting stop time according to other embodiments of the present disclosure.

In some embodiments, as shown in FIG. 5, the processor determines an area change rate 530 based on a first area 510 and a second area 520, the first area 510 being an area of a first zone, the second area 520 being an area of a second zone; determining the water level change rates 540 for the plurality of monitoring wells; and determining a grouting stop time 550 for a current batch of a plurality of grouting holes based on the water level change rates 540 and the area change rate 530.

For more descriptions regarding the first area, the second area, the first zone, and the second zone, refer to FIG. 1 and the related descriptions.

The area change rate refers to a change rate of the second area relative to the first area. The area change rate may measure whether the grouting operation is effective. For example, if the area change rate is a positive count, it indicates that the grouting is effective; otherwise, it is ineffective.

In some embodiments, the processor may determine the area change rate based on a relationship where the area change rate is positively correlated with an area difference between the second area and the first area and negatively correlated with a grouting duration. Merely by way of example:

$${Δ}_{s} = \frac{{s}_{2} - {s}_{1}}{{Δ}_{t}} , \qquad ( 3 ) ,$$

where in Equation (3), Δₛ denotes the area change rate, s₂ denotes the second area, s₁ denotes the first area, and Δₜ denotes the grouting duration.

The grouting duration refers to a duration reached by the grouting operation.

The water level change rate refers to a change rate of a groundwater level in the monitoring well before and after grouting. The water level change rate can reflect whether the grouting operation successfully prevents the ground surface water from infiltrating underground.

In some embodiments, the processor may determine the water level change rate based on a relationship where the water level change rate is positively correlated with a water level difference between a water level after grouting and a water level before grouting, and negatively correlated with the grouting duration. Merely by way of example:

$${Δ}_{l} = \frac{{l}_{2} - {l}_{1}}{{Δ}_{t}} , \qquad ( 4 ) ,$$

where in Equation (4), Δₜ denotes the water level change rate, l₂ denotes the water level after grouting, l₁ denotes the water level before grouting, and Δₜ denotes the grouting duration.

The water level before grouting refers to an initial water level height in the monitoring well before the grouting operation. The water level after grouting refers to a stable water level height in the monitoring well after the grouting operation.

The processor may measure the water level before grouting and the water level after grouting based on a water level sensor in the monitoring well.

The grouting stop time characterizes a duration for which grouting needs to continue from the current moment. For example, if the grouting stop time is 2 hours, it indicates that grouting may be terminated after 2 hours.

In some embodiments, the processor determines a first grouting stop time based on a relationship where the first grouting stop time is positively correlated with an area threshold and negatively correlated with a current area and the area change rate. Merely by way of example:

$${t}_{s} = \frac{{s}_{0} - {s}_{3}}{{Δ}_{s}} , \qquad ( 5 ) ,$$

where in Equation (5), tₛ denotes the first grouting stop time, s₃ denotes the current area, so denotes the area threshold, and Δₛ denotes the area change rate.

The current area refers to an area of a dominant water-conducting channel zone (e.g., the first dominant water-conducting channel zone or the second dominant water-conducting channel zone) in the current grouting operation.

In some embodiments, the processor determines a second grouting stop time based on a relationship where the second grouting stop time is positively correlated with a water level threshold and negatively correlated with the water level change rate and a current water level. Merely by way of example:

$${t}_{1} = \frac{{l}_{0} - {l}_{3}}{{Δ}_{l}} , \qquad ( 6 ) ,$$

where in Equation (6), t₁ denotes the second grouting stop time, l₀ denotes the water level threshold, l₃ denotes the current water level, and Da denotes the water level change rate.

The area threshold and the water level threshold may be obtained by presetting.

In some embodiments, if the area change rate is a positive value and a water level change is greater than or equal to a water level change threshold, it indicates that the current grouting operation is effective, and grouting will be stopped after the grouting stop time. If the area change rate is a negative value or zero and the water level change is less than the water level change threshold, it indicates that the current grouting operation is ineffective, and grouting should be stopped immediately to avoid resource waste.

In some embodiments, the processor determines the grouting stop times for the grouting holes in a plurality of directions based on boundary change rates in the plurality of directions and water level change rates in the plurality of directions.

For descriptions regarding the directions, please refer to FIG. 3 and the related descriptions.

The boundary change rate is a change rate of the dominant water-conducting channel zone at different times.

In some embodiments, for each direction, the processor may determine the boundary change rate based on a relationship where the boundary change rate is positively correlated with a boundary reduction value at a second time and negatively correlated with a boundary reduction value at a first time and a time interval. Merely by way of example:

$${Δ}_{a} = \frac{{a}_{2} - {a}_{1}}{{T}_{1}} , \qquad ( 7 ) ,$$

where in Equation (7), Δₐ denotes the boundary change rate, a₂ denotes the boundary reduction value at the second time, a₁ denotes the boundary reduction value at the first time, and T₁ denotes the time interval.

The second time is after the first time and may be obtained by presetting. The time interval refers to a difference between the second time and the first time.

In some embodiments, the processor may determine the grouting stop times for the grouting holes in a plurality of directions based on the boundary change rates in the plurality of directions and the water level change rates in the plurality of directions in various manners.

In some embodiments, for each direction, the processor may determine a third grouting stop time based on the boundary change rate in the direction. The processor may determine a fourth grouting stop time based on the water level change rate in the direction. The processor may select a later time of the third grouting stop time and the fourth grouting stop time as a final determined grouting stop time.

The third grouting stop time and the fourth grouting stop time are similar to the grouting stop time. The above expressions are used for ease of distinction.

In some embodiments, for each direction, the processor determines the third grouting stop time based on a relationship where the third grouting stop time is positively correlated with a distance difference and negatively correlated with a boundary threshold and the boundary change rate. Merely by way of example:

$${t}_{a} = \frac{v - {a}_{0}}{{Δ}_{a}} , \qquad ( 8 ) ,$$

where in Equation (8), tₐ denotes the third grouting stop time, v denotes the distance difference, a₀ denotes the boundary threshold, and Δₐ denotes the boundary change rate.

The distance difference refers to a distance between a boundary point of the dominant water-conducting channel zone and a center point of the dominant water-conducting channel zone in a currently selected direction.

The boundary threshold may be obtained by presetting.

In some embodiments, for each direction, a manner for determining the fourth grouting stop time is similar to a manner for determining the second grouting stop time, which is not repeated herein.

In some embodiments, for each direction, if the boundary change rate is a negative value and the water level change rate is greater than or equal to a water level change threshold, it indicates that the current grouting operation is effective, and grouting will be stopped after the grouting stop time. If an area change rate is a negative value or zero, and the water level change rate is less than the water level change threshold, it indicates that the current grouting operation is ineffective, and grouting should be stopped immediately to avoid resource waste. The water level change threshold may be obtained by presetting.

By predicting the grouting stop time direction by direction, a grouting strategy may be accurately adjusted for local weak areas, avoiding a one-size-fits-all approach globally. This improves resource utilization and grouting efficiency.

In some embodiments, the processor may further determine the grouting updating parameters based on the grouting stop time for the plurality of grouting holes and a grouting time for the plurality of grouting holes, and control the automatic grouting device to perform the grouting operation on a corresponding grouting hole according to the grouting updating parameters.

In some embodiments, the processor compares the grouting stop time of the plurality of grouting holes with the grouting time of the plurality of grouting holes. If a time interval between the grouting stop time of a grouting hole among the plurality of grouting holes and the grouting time of the grouting hole is less than a time threshold, the grouting parameters corresponding to the grouting hole need to be adjusted.

Manners for determining the grouting updating parameters and performing the grouting operation based on the grouting updating parameters are similar to step 340 described above, and are not repeated herein.

By comparing the grouting time and the grouting stop time in real time, adjustment of the grouting parameters is automatically triggered when the time interval is less than the time threshold. This effectively avoids over-grouting caused by untimely control, thereby reducing material waste, lowering the risk of device damage, and improving the accuracy and safety of grouting control.

By collecting the monitoring data and the apparent resistivity in real time, the grouting effect is dynamically determined, and the grouting updating parameters are determined. This transforms the grouting process from a static operation to data-driven intelligent control. This solves the lag and uncertainty problems of traditional post-event evaluation, improves the efficiency and accuracy of the grouting operation, and effectively reduces grouting cost.

Based on the area change rate and the water level change rate, the grouting stop time is determined. This achieves a shift from empirical judgment to data-driven intelligent control, effectively avoiding over-grouting or ineffective construction, reducing cost, and improving the water retention effect.

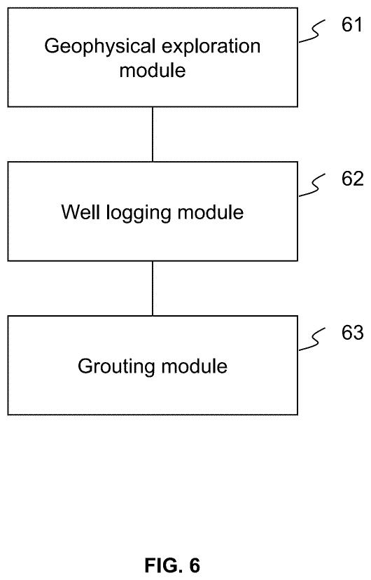

FIG. 6 is a schematic diagram illustrating a structure of a device for reconstructing a water-retaining and water-blocking layer by grouting in a mining area according to some embodiments of the present disclosure.

In some embodiments of the present disclosure, a device for reconstructing a water-retaining and water-blocking layer by grouting in a mining area is provided. Referring to FIG. 6, the device includes: a geophysical exploration module 61, configured to perform the geophysical exploration from a aquifer to a goaf in a coal mining remediation zone of the mining area to obtain a first apparent resistivity at each of positions within a space, and to select positions within the space where the first apparent resistivity is less than or equal to a first preset resistivity to form a first dominant water-conducting channel zone; a well logging module 62, configured to perform a water flow direction test in at least two monitoring wells within the coal mining remediation zone to obtain a first flow direction fitting function for each monitoring well of the at least two monitoring wells, and to select two first flow direction fitting functions to calculate a first flow direction intersection corresponding to the two monitoring wells; and a grouting module 63, configured to determine that the first dominant water-conducting channel zone is accurate when the first flow direction intersection is located within the first dominant water-conducting channel zone, and perform a grouting operation on the first dominant water-conducting channel zone.

The geophysical exploration module refers to an intelligent unit configured to perform the geophysical exploration and obtain and analyze results of the geophysical exploration. The geophysical exploration module may include a main control detector, a sensor array, a data processing server, etc.

The well logging module refers to a core functional unit configured to obtain underground water flow dynamic information through the monitoring wells and thereby verify the accuracy of the results of the geophysical exploration. The well logging module may include a downhole flowmeter, a data logger, etc.

The grouting module refers to an intelligent execution unit responsible for performing a final grouting operation. The grouting module may include an intelligent grouting pump station, grouting pipelines and valve groups, etc.

The device of the above embodiment is used to implement the method for reconstructing the water-retaining and water-blocking layer by grouting in the mining area corresponding to any of the foregoing embodiments. The device has beneficial effects corresponding to the method embodiments, which are not repeated herein.

Based on the same inventive concept and corresponding to any of the above method embodiments, the present disclosure further provides an electronic device, including: a memory, a processor, and a computer program stored in the memory and executable on the processor, and the processor, when executing the computer program, implements the method for reconstructing the water-retaining and water-blocking layer by grouting in the mining area.

FIG. 7 is a schematic diagram illustrating a structure of the electronic device according to some embodiments of the present disclosure.

In some embodiments, as shown in FIG. 7, the electronic device includes a memory 7020, a processor 7010, and a computer program stored in the memory 7020 and executable on the processor 7010. When the processor 7010 executes the computer program, the foregoing method for reconstructing the water-retaining and water-blocking layer by grouting in the mining area is implemented.

In some embodiments, the electronic device may further include an input/output interface 7030, a communication interface 7040, and a bus 7050. The processor 7010, the memory 7020, the input/output interface 7030, and the communication interface 7040 are communicatively connected to each other within the device via the bus 7050.

The processor 7010 may be implemented by a general-purpose central processing unit (CPU), a microprocessor, an application-specific integrated circuit (ASIC), or one or more integrated circuits. The processor 7010 is configured to execute related programs to implement the technical solutions provided by the embodiments of the present disclosure.

The memory 7020 may be implemented by a read-only memory (ROM), a random access memory (RAM), a static storage device, a dynamic storage device, etc. The memory 7020 may store an operating system and other application programs. When the technical solutions provided by the embodiments of the present disclosure are implemented by software or firmware, related program codes are stored in the memory 7020 and called and executed by the processor 7010.

In some embodiments, the processor may be integrated into the geophysical exploration module, the well logging module, and the grouting module.

The input/output interface 7030 is configured to connect to an input/output module to implement information input and output. The input/output module (also referred to as an input device/output device) may be configured as a component in the device (not shown in FIG. 7) or may be externally connected to the device to provide corresponding functions. The input device may include a keyboard, a mouse, a touch screen, a microphone, various types of sensors, etc. The output device may include a display, a speaker, a vibrator, an indicator light, etc.

The communication interface 7040 is configured to connect to a communication module (not shown in FIG. 7) to implement communication interaction between the device and other devices. The communication module is characterized in that it may implement communication through a wired manner (e.g., USB, network cable) or through a wireless manner (e.g., mobile network, WIFI, Bluetooth).

The bus 7050 includes a pathway configured to transmit information among various components (e.g., the processor 7010, the memory 7020, the input/output interface 7030, and the communication interface 7040) of the device.

It should be noted that although the above device only shows the processor 7010, the memory 7020, the input/output interface 7030, the communication interface 7040, and the bus 7050, in a specific implementation, the device may further include other components necessary for normal operation. Furthermore, those skilled in the art may understand that the above device may also include only components necessary for implementing the solutions of the embodiments of the present disclosure, and does not necessarily have to include all components shown in the figure.

The electronic device of the above embodiment is used to implement the method for grouting and water retention in the coal mining remediation zone corresponding to any of the foregoing embodiments. The electronic device has beneficial effects corresponding to the method embodiments, which are not repeated herein.

It should be noted that the above describes some embodiments of the present disclosure. Other embodiments are within the scope of the appended claims. In some cases, the actions or steps recited in the claims may be performed in an order different from that in the above-described embodiments and still achieve the desired results. In addition, the processes depicted in the accompanying drawings do not necessarily require the particular order or sequential order shown to achieve the desired results. In certain implementations, multitasking and parallel processing are also possible or may be advantageous.

Those of ordinary skill in the art should understand that the discussion of any of the above embodiments is merely exemplary and is not intended to imply that the scope of the present disclosure, including the claims, is limited to these examples. Under the concept of the present disclosure, the technical features in the above embodiments or in different embodiments may also be combined, the steps may be implemented in any order, and there are many other variations of different aspects of the embodiments of the present disclosure as described above, which are not provided in detail for the sake of brevity.

In addition, to simplify the description and discussion, and to avoid making the embodiments of the present disclosure difficult to understand, well-known power/ground connections to other components may or may not be shown in the provided drawings. Furthermore, the devices may be shown in block diagram form to avoid making the embodiments of the present disclosure difficult to understand, and this also considers the fact that the details of the implementation of these block diagram devices are highly dependent on the platform on which the embodiments of the present disclosure are to be implemented (i.e., these details should be entirely within the understanding of those skilled in the art). While specific details have been set forth to describe exemplary embodiments of the present disclosure, it will be apparent to those skilled in the art that the embodiments of the present disclosure may be practiced without these specific details or with variations of these specific details. Therefore, these descriptions should be considered as illustrative and not restrictive.

Although the present disclosure has been described in connection with specific embodiments of the present disclosure, many alternatives, modifications, and variations of these embodiments will be apparent to those of ordinary skill in the art from the foregoing description. The embodiments of the present disclosure are intended to cover all such alternatives, modifications, and variations that fall within the broad scope of the appended claims. Therefore, any omissions, modifications, equivalent substitutions, improvements, etc., made within the spirit and principles of the embodiments of the present disclosure shall be included within the protection scope of the present disclosure.

What is claimed is:

1. A method for reconstructing a water-retaining and water-blocking layer by grouting in a mining area, comprising:
performing geophysical exploration within a space from an aquifer to a goaf in a coal mining remediation zone of a mining area to obtain a first apparent resistivity at each of positions within the space, and selecting positions within the space where the first apparent resistivity is less than or equal to a first preset resistivity to form a first dominant water-conducting channel zone;
performing a water flow direction test in at least two monitoring wells within the coal mining remediation zone to obtain a first flow direction fitting function for each monitoring well of the at least two monitoring wells, and selecting two first flow direction fitting functions to calculate a first flow direction intersection corresponding to two monitoring wells; and
determining that the first dominant water-conducting channel zone is accurate when the first flow direction intersection is located within the first dominant water-conducting channel zone, and performing a grouting operation on the first dominant water-conducting channel zone.

2. The method according to claim 1, further comprising:
increasing the first preset resistivity to reform the first dominant water-conducting channel zone when the first flow direction intersection is located outside the first dominant water-conducting channel zone, and subsequently using the water flow direction test to verify again whether the first dominant water-conducting channel zone is accurate.

3. The method according to claim 1, further comprising:
obtaining geological parameters of the coal mining remediation zone, and determining a water-filling source based on the geological parameters; and
performing geophysical exploration from a ground surface to the goaf in the coal mining remediation zone when the water-filling source is from a Quaternary aquifer.

4. The method according to claim 1, further comprising:
after performing the grouting operation, performing geophysical exploration again within the space from the aquifer to the goaf in the coal mining remediation zone to obtain a second apparent resistivity at each of the positions within the space; selecting positions within the space where the second apparent resistivity is less than or equal to the first preset resistivity to form a second dominant water-conducting channel zone; and
stopping grouting when the second dominant water-conducting channel zone is located within the first dominant water-conducting channel zone.

5. The method according to claim 1, wherein after obtaining the first apparent resistivity at each of the positions within the space, the method comprises: selecting positions within the aquifer where the first apparent resistivity is less than or equal to the first preset resistivity to form a first zone, an area of the first zone being a first area; and
the method further comprises: after performing the grouting operation, performing geophysical exploration again within the space from the aquifer to the goaf in the coal mining remediation zone to obtain a second apparent resistivity at each of the positions within the space; selecting positions within the aquifer where the second apparent resistivity is less than or equal to the first preset resistivity to form a second zone, an area of the second zone being a second area; and stopping grouting when the second area is greater than the first area.

6. The method according to claim 5, further comprising:
when the second area is greater than the first area, a volume of a second dominant water-conducting channel zone is less than or equal to a volume of the first dominant water-conducting channel zone multiplied by a first preset ratio, and a second flow direction intersection is located outside the first dominant water-conducting channel zone, stopping grouting.

7. The method according to claim 5, further comprising:
adjusting borehole positions of a subsequent batch of grouting holes based on a difference between a characteristic of a second dominant water-conducting channel zone after performing the grouting operation using a previous batch of grouting holes and a characteristic of the first dominant water-conducting channel zone.

8. The method according to claim 1, further comprising:
after performing the grouting operation, performing the water flow direction test again in the at least two monitoring wells within the coal mining remediation zone to obtain a second flow direction fitting function for the each monitoring well, and selecting two second flow direction fitting functions to calculate a second flow direction intersection corresponding to two monitoring wells; and
stopping grouting when the second flow direction intersection is located outside the first dominant water-conducting channel zone.

9. The method according to claim 1, further comprising:
after a preset time period following completion of grouting, performing a water level test in a monitoring well within the coal mining remediation zone to obtain a first water level drop, and performing the water level test in a monitoring well outside the coal mining remediation zone to obtain a second water level drop; and
completing water retention when the first water level drop is less than or equal to the second water level drop multiplied by a second preset ratio.

10. The method according to claim 1, further comprising:
obtaining monitoring data collected and uploaded by a plurality of sensors, the plurality of sensors being deployed in a plurality of grouting holes and/or a plurality of monitoring wells;
collecting an apparent resistivity at a preset position through geophysical exploration;
determining a grouting effect based on the monitoring data and the apparent resistivity; and
determining grouting updating parameters based on the grouting effect, and controlling an automatic grouting device to perform the grouting operation on a corresponding grouting hole according to the grouting updating parameters.

11. The method according to claim 10, further comprising:
determining the plurality of grouting holes and a grouting sequence based on the first dominant water-conducting channel zone and the first flow direction intersection;
controlling the automatic grouting device to perform the grouting operation on a first batch of grouting holes based on the grouting sequence;
in response to determining that a grouting effect corresponding to the first batch of grouting holes satisfies a grouting stop condition, stopping grouting; or
in response to determining that the grouting effect corresponding to the first batch of grouting holes does not satisfy the grouting stop condition, updating, based on a grouting effect of a previous batch of grouting holes, a grouting sequence and grouting parameters for a subsequent batch of grouting holes to obtain an updated grouting sequence and updated grouting parameters, and controlling the automatic grouting device to perform the grouting operation according to the updated grouting sequence and the updated grouting parameters.

12. The method according to claim 11, further comprising:
determining the first batch of grouting holes based on a distance between each grouting hole of the plurality of grouting holes and the first flow direction intersection and a shortest distance between the each grouting hole and a boundary of the first dominant water-conducting channel zone.

13. The method according to claim 1, further comprising:
determining an area change rate based on a first area and a second area, the first area being an area of a first zone, the second area being an area of a second zone;
determining water level change rates for a plurality of monitoring wells; and
determining a grouting stop time for a current batch of a plurality of grouting holes based on the water level change rates and the area change rate.

14. The method according to claim 13, further comprising:
determining grouting stop times for grouting holes in a plurality of directions based on boundary change rates in the plurality of directions and water level change rates in the plurality of directions.

15. The method according to claim 13, further comprising:
determining grouting updating parameters based on the grouting stop time for the plurality of grouting holes and a grouting time for the plurality of grouting holes, and controlling an automatic grouting device to perform the grouting operation on a corresponding grouting hole according to the grouting updating parameters.

16. A device for reconstructing a water-retaining and water-blocking layer by grouting in a mining area, using the method for reconstructing a water-retaining and water-blocking layer by grouting in a mining area according to claim 1, comprising:
a geophysical exploration module, configured to perform the geophysical exploration from the aquifer to the goaf in the coal mining remediation zone of the mining area to obtain the first apparent resistivity at the each of positions within the space, and to select the positions within the space where the first apparent resistivity is less than or equal to the first preset resistivity to form the first dominant water-conducting channel zone;
a well logging module, configured to perform the water flow direction test in the at least two monitoring wells within the coal mining remediation zone to obtain the first flow direction fitting function for the each monitoring well of the at least two monitoring wells, and to select the two first flow direction fitting functions to calculate the first flow direction intersection corresponding to two monitoring wells; and
a grouting module, configured to determine that the first dominant water-conducting channel zone is accurate when the first flow direction intersection is located within the first dominant water-conducting channel zone, and perform the grouting operation on the first dominant water-conducting channel zone.

17. An electronic device, comprising a memory, a processor, and a computer program stored in the memory and executable on the processor, wherein the processor, when executing the computer program, implements the method for reconstructing a water-retaining and water-blocking layer by grouting in a mining area according to claim 1.
